(19) United States
(12) Patent Application Publication
(10) Pub. No.: US 2006/0184105 A1
(43) Pub. Date: Aug. 17, 2006

(54) Thin wall catheter and method of placing same

(76) Inventors: Gregory L. Townsend, Plymouth, MN (US); Thomas M. Soukup, Plymouth, MN (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(21) Appl. No.: 11/058,685

(22) Filed: Feb. 15, 2005

(51) Int. Cl. A61M 31/00

(52) U.S. Cl. 604/93.01; 604/158; 606/108

(57) ABSTRACT

Methods and apparatus for introducing diagnostic and/or therapeutic agents and devices within a mammalian body are disclosed. One disclosed apparatus comprises a sheath having a proximate end, a distal end and a lumen extending therebetween. An articulator is disposed in the lumen of the sheath. A distal portion of the sheath is detachably attached to a distal portion of the articulator at a joint. A separator capable of severing the joint is also provided. One disclosed method comprises the step of providing a device comprising a sheath disposed about an articulator with a distal portion of the sheath being fixed to the articulator at a joint. The device is inserted into a lumen of a body. The joint is severed and the articulator is withdrawn from the sheath.

### TECHNICAL FIELD

The present disclosure relates generally to the introduction of diagnostic and/or therapeutic agents and devices within a mammalian body.

### BACKGROUND OF THE INVENTION

The present disclosure relates generally to the field of introducing diagnostic and therapeutic agents and devices within the human body. By placing a hollow tube in a patient's body a surgeon may gain access to difficult to reach areas of the anatomy. The patient has great benefit from not having large muscles and nerves severed by the surgeon to gain access to the afflicted area. This is sometimes referred to minimally invasive forms of surgery.

The catheter was invented 95 years ago by Charles Russell Bard for the treatment of urinary discomfort. Since that time, many special purpose catheters have been developed. A great deal of engineering effort has gone into making catheters rigid and resistant to kinking. This rigidity and kink resistance aids in the ability to advance the catheter through the vasculature. A very stiff catheter may have a difficult time following in the tortuous conduits of the human vasculature system without tending to straighten the vessels to conform to the tubing. Additionally, the presence of the catheter in the vasculature restricts blood flow. The stiffness of the catheter may cause damage to the walls of blood vessels. An example of this adverse effect is a clot that can travel to the lungs and cause death by pulmonary embolism. Pulmonary embolism is an obstruction of a blood vessel in the lungs which blocks a coronary artery. According to the American Heart Association, an estimated 600,000 Americans develop pulmonary embolisms annually.

Catheters are often manufactured with rigid walls in order to facilitate pushing of the catheter over the guidewire. Physicians sometimes complain that forcing catheters with heavy walls through the veins and arteries cause damage to the vascular endothelium. In addition, clots can form at the tips of these catheters.

Stylets and guidewires are used to control the manner in which intravascular leads and catheters are introduced into the veins or arteries of the body. Conventional intravascular procedures typically involve an initial step of introducing and routing a guidewire through a patient's vascular system to provide a rail or track along which additional devices may be introduced. Once a guidewire is in place, a catheter is routed over at least a portion of the guidewire to provide a larger opening into the vein or artery and sometimes to protect the inside walls of the vessels along the route of the guidewire. With the sheath in place, the guidewire may be removed or may remain in place as additional devices such as intravascular leads and catheters are introduced into the patient's vascular system.

In contrast to the guidewire which serves as a track over which other devices are routed, a stylet is used within an internal lumen of a device both to push that device through the vascular system and to steer the device as it is being pushed. Although some devices are designed to steer themselves using internal pull wires, almost all leads, most catheters and some guidewires have an inner channel or lumen into which a stylet is inserted. In addition to pushing the device through the vascular system by engaging the distal end of the device, the stylet also serves to deflect the distal end of the device so as to steer the distal end through the vascular system. Unlike the lead, catheter or guidewire, which has a distal region that is flexible and floppy, the stylet must be stiffer and more rigid so as to enable the stylet to push the lead or catheter through the patient's vascular system. Stylets have been provided with steerable technology, such as described in applicants own U.S. application Ser. No. 09/934,245, filed Aug. 21, 2001, the contents of which are herein incorporated by reference. Further, stylets have been provided with adjustable stiffness technology, as described in applicant's own U.S. application Ser. No. 09/843,040, filed Apr. 25, 2001, the contents of which are herein incorporated by reference. Such steerable and adjustable technologies are useful for guiding the stylet and/or device through the vascular system.

The more control and flexibility an operator has over a device, the easier it is to operate that device. In the case of stylets, the physical demands of engaging the distal end of a lumen of a device so as to push that device through the vascular system impose constraints on the beam strength of the device that are much different than the constraints encountered for a guidewire, catheter or lead. Most guidewires are constructed from a tapered core wire with a coiled round wire wrapped around this tapered core wire in order to achieve the necessary flexibility in the distal region of the guidewire. Stylets, on the other hand, are generally constructed of a solid wire of uniform diameter without any coils around this wire in order to achieve the necessary strength and rigidity required over the entire length of the device so as to function as a stylet.

The flexibility of the stylet, as well as its ability to appropriately guide the device through a vascular channel is currently compromised by its engagement with the distal end of the device. For example, the stylet may currently be retained within the distal end of a catheter. Therefore, when the stylet is guided, such as by steering it, it must articulate within the catheter. In such a system the catheter will also articulate, which produces greater stress on the steerable portion of the stylet and reduces the ability of the device to be steered within the vascular system. Therefore, a need exists for a device that couples a stylet with a medical device such as a catheter in such a way that the stylet's ability to guide the device through the body is not compromised, yet which allows the stylet to be detached from the device and retracted from the body, leaving the device in place.

### SUMMARY OF THE INVENTION

The present disclosure relates generally to the introduction of diagnostic and/or therapeutic agents and devices within the human body. A device in accordance with the present invention comprises the articulator and a sheath. In some advantageous embodiments, the articulator is a steerable articulator. Also in some advantageous embodiments, the articulator comprises one or more adjustable stiffness sections. In one aspect of the present invention, the sheath comprises a relatively thin wall. This thin wall, maximizes the inner diameter of the sheath allowing larger objects to pass through a lumen defined by the sheath.

A distal portion of the sheath is attached to a distal portion if the articulator. The sheath is carried to the site of therapy by the articulator and then released by the motion of sliding tubular member surrounding the articulator. The sheath may be constructed from polymer materials that are proven to be hemo-compatible for limited exposure, prolonged exposure and/or permanent contact. Limited exposure generally means that the human body is exposed to the device for less than 24 hours. Prolonged exposure generally means that the human body is exposed to the device for between 24 hours and 30 days. Permanent contact generally means that the human body is exposed to the device for between 24 hours and 30 days. A proximal portion of the articulator resides loose within a lumen defined by the sheath to allow the sheath and the articulator to flex independently.

The sheath differs from existing catheters by virtue of a thin wall and flexibility. The thin wall allows blood to flow more easily around and along the length of the sheath. The sheath may be soft enough that the arterial pressure will collapse the thin wall causing little occlusion to the blood flow. The sheath may remain collapsed until a secondary device is passed through the sheath lumen. The lumen will open to allow the device to pass but will still allow maximum blood flow. A sheath in accordance with the present invention may have a hoop strength that is selected so that the catheter will collapse while it is disposed within a blood vessel. The blood pressure causes the sheath to collapse. The blood pressure may be for example 60 millimeters of mercury.

In some embodiments, the sheath may comprise a distal section that is of a different durometer than other portions of the sheath. This different durometer section may function to hold the distal end proximate a target location in the body. This different durometer section may also function to strengthen the connection between the sheath and the articulator. The different durometer section may be an elongated section of a few millimeters comprising a harder material. The different durometer section may comprise an O-ring. A lure fitting may be coupled to a proximate end of the sheath.

The sheath may comprise a straight tube as one example. The sheath may comprise a tube with two or more lumens as another example. When this is the case, the articulator may be placed in the smaller of the two lumens. The sheath may be pulled to the target site by the articulator. The articulator may then be retracted a distance to allow the distal catheter tube to collapse when the physician inserts another medical device into the larger of the two lumens. In some cases, the articulator may be partially retracted without drawing the articulator completely out of the lumen. When this is the case, the articulator need only be retracted far enough so as not to interfere with the other devices being passed through the sheath.

In some embodiments, the invention relates to a device that includes a medical device comprising a sheath and the articulator. The articulator and the sheath may be operatively connected proximate their distal ends. A deployment actuator may be provided to detach the sheath and the articulator. In some embodiments, the deployment actuator comprises a tube with a length longer than that of the sheath. When the tube is pushed in the distal direction it breaks a bond between the articulator and the sheath. In such embodiments, the articulator retains its ability to guide the device through the body. The invention also includes methods of placing diagnostic and therapeutic agents and devices inside a body.

The deployment apparatus described above may allow for a sheath with a thinner wall than conventional catheters because the stiffness of the articulator allows the sheath to be placed without relying on its own rigidity. Such a thin wall sheath is advantageous because it allows for maximization of the lumen diameter to allow for the passage of larger devices through the sheath given the same outside diameter size. The larger devices passing through a protective sheath may cause less vascular disruption than the larger device being pushed through the vessel by itself. Such embodiments are useful for reducing damage to the vascular system during intravascular procedures. In many procedures done today, the larger device (e.g., PCTA balloon catheter) is pushed over a guidewire. PCTA catheters are typically quite stiff and have uneven surfaces that may cause disruption to the vascular system.

### BRIEF DESCRIPTION OF THE DRAWINGS

FIG. 1 shows a perspective view of a device in accordance with an embodiment of the invention.

FIG. 2 shows a perspective view of an alternate embodiment of a device in accordance with an embodiment of the invention.

FIG. 3 shows a perspective view of an embodiment of a device in accordance with an embodiment of the invention.

FIG. 4 shows an end view of a medical device in accordance with an embodiment of the present invention.

FIG. 5 shows a perspective view of an adjustable steering stylet in accordance with an embodiment of the present invention.

FIG. 6 shows a perspective view of an adjustable stiffness stylet in accordance with an embodiment of the present invention.

FIG. 7 is a cross sectional view of a device comprising an articulator and a sheath.

FIG. 8 is an additional cross sectional view of the device shown in the previous figure.

FIG. 9 is an additional cross sectional view of the device shown in the previous figure.

FIG. 10 is an additional cross sectional view of sheath shown in the previous figure.

FIG. 11 is ventral view of a patient and a device in accordance with an exemplary embodiment of the present invention.

FIG. 12 is a diagrammatic view illustrating vasculature system shown in the previous figure.

FIG. 13 is an additional diagrammatic view of device and vasculature system shown in the previous figure.

FIG. 14 is an additional diagrammatic view of device and vasculature system shown in the previous figure.

FIG. 15 is a partial cross sectional view of a device comprising an articulator and a sheath.

FIG. 16 is an additional cross sectional view of the device shown in the previous figure.

FIG. 17 is a cross sectional view of a sheath in accordance with the present invention.

FIG. 18 is an isometric view of a sheath in accordance with an exemplary embodiment of the present invention.

FIG. 19 is an isometric view of an assembly including the sheath shown in the previous figure.

FIG. 20 is an axial view of the sheath shown in the previous figures.

FIG. 21 is an additional axial view showing the sheath shown in the previous figure.

FIG. 22 is an additional axial view showing the sheath shown in the previous figure.

FIG. 23 is a cross-sectional view of a sheath in accordance with an exemplary embodiment of the present invention.

FIG. 24 is a cross-sectional view of a sheath in accordance with an additional exemplary embodiment of the present invention.

FIG. 25 is a cross sectional view of a device comprising an articulator and a sheath.

FIG. 26 is an additional cross sectional view of the device shown in the previous figure.

FIG. 27 is an additional cross sectional view of the device shown in the previous figure.

FIG. 28 is an additional cross sectional view of the sheath shown in the previous figure.

FIG. 29 is a partial cross sectional view of a device comprising an articulator and a sheath.

FIG. 30 is an additional cross sectional view of the device shown in the previous figure.

### DETAILED DESCRIPTION

The following detailed description should be read with reference to the drawings, in which like elements in different drawings are numbered identically. The drawings, which are not necessarily to scale, depict selected embodiments and are not intended to limit the scope of the invention. Examples of constructions, materials, dimensions, and manufacturing processes are provided for selected elements. All other elements employ that which is known to those of skill in the field of the invention. Those skilled in the art will recognize that many of the examples provided have suitable alternatives that can be utilized.

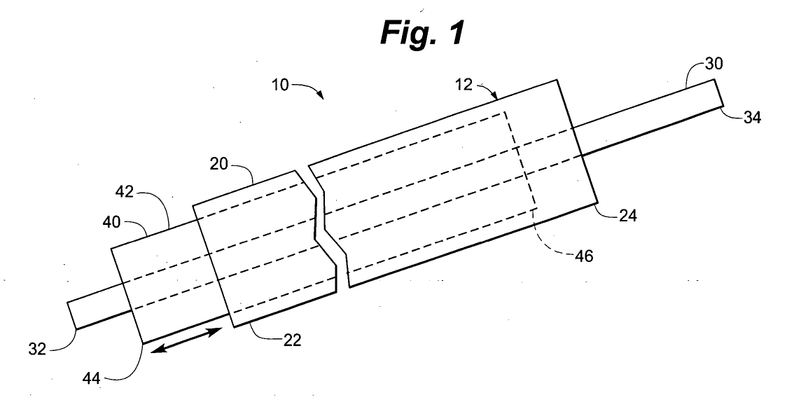

FIG. 1 shows a device 10 in accordance with the present invention. Device 10 may include a medical device 12. Medical device 12 may be any device capable of placement within a vascular system of a mammalian body. In some embodiments, medical device 12 comprises a sheath 20 having a proximal end 22 and a distal end 24. Sheath 20 may comprise a lubricious coating on its outside and/or inside surfaces. Further, sheath 20 may comprise a relatively thin wall. A relatively thin wall provides a larger lumen size for a given outside diameter or the same lumen size for a smaller outside diameter. The increased lumen size is useful for passing larger devices through the sheath 20 while minimizing damage to the vascular system.

Device 10 also may include an articulator 30 useful for the placement of the sheath 20 within a vascular system and having a proximal end 32 and a distal end 34. The articulator 30 may be coated with a lubricious coating. In some embodiments, the articulator 30 comprises one or more steerable poritons and/or one or more adjustable stiffness portions.

Device 10 may also include a deployment actuator 40 useful for detaching the sheath 20 and the articulator 30 once the device 10 is placed within the vascular system. The detachment of sheath 20 and articulator 30 allows the articulator 30 to be retracted from the body, leaving the sheath 20 in place. Thereafter, other tools may be placed into the lumen of the sheath 20. Deployment actuator 40 may be any device capable of detaching sheath 20 and articulator 30 from each other, and may contain a lubricous coating on its outside and/or inside surfaces.

In some embodiments, the deployment actuator 40 may comprise a tube 42 having a proximal end 44 and a distal end 46. Tube 42 may comprise any material capable of flexing though a vascular system and of transmitting axial force sufficient to detach sheath 20 from articulator 30. In some embodiments, tube 42 comprises a polymeric material such as polyurethane and/or polytetrafluoroethylene.

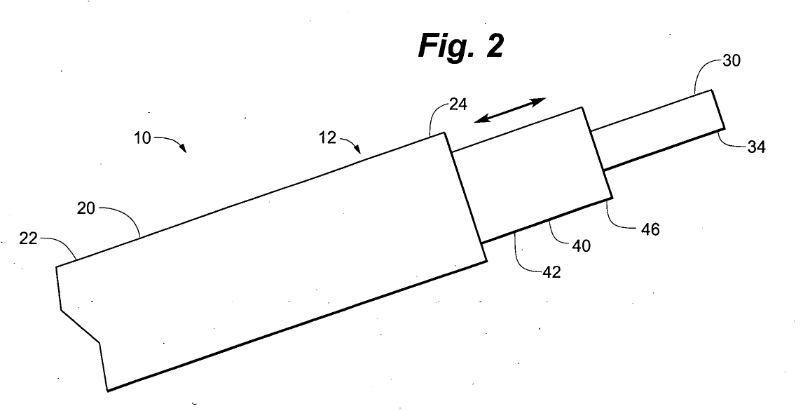

Tube 42 may be allowed to slide axially between the sheath 20 and the articulator 30 as shown in FIG. 1. In such an embodiment, moving the tube 42 in a distal direction will detach the sheath 20 from the articulator 30. In other embodiments, sheath 20 and articulator 30 are both attached to tube 42 as shown in FIG. 2. In such an embodiment, moving tube 42 in either a proximate or distal direction will functionally detach sheath 20 from articulator 30.

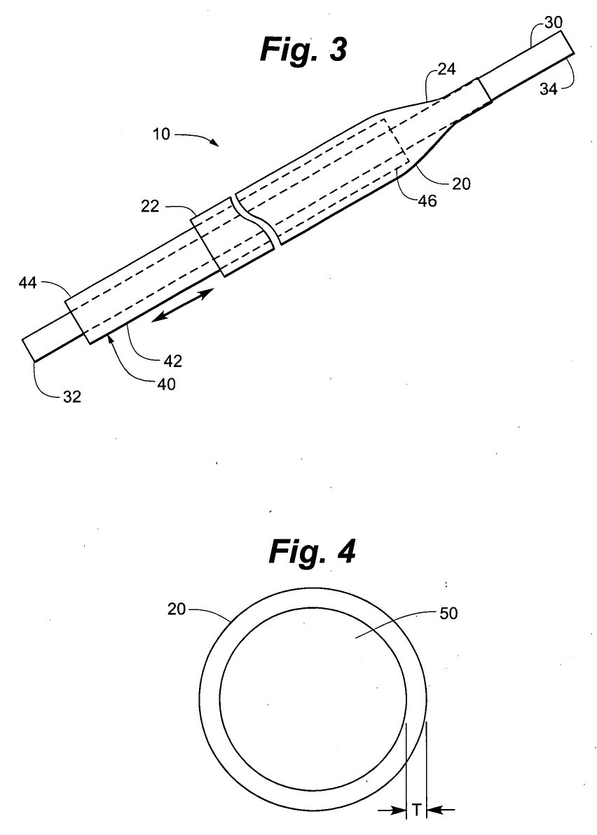

Sheath 20 and articulator 30 may be operatively coupled together in any fashion. For example, sheath 20 and articulator 30 may be operatively coupled together by a vacuum seal, adhesive, chemical bond, and/or mechanical linkage. An embodiment of a vacuum seal is shown in FIG. 3, where the distal end 24 of the sheath 20 is pulled around the articulator 30. Moving the tube 42 in the distal direction breaks this seal. An adhesive bond may comprise various adhesives without deviating from the spirit and scope of the present invention. Adhesives that may be suitable in some applications may include adhesives cyanoacrylate adhesives, polyethylene glycol adhesives, silicone adhesives, fibrin sealants, albumin sealants

Such a deployment system allows for the catheter to have a thin wall compared to conventional catheters, thereby providing a larger lumen 50 as shown in FIG. 4. Such a large lumen 50 is possible because the walls of the sheath 20 need not be as rigid as the walls of a conventional catheter. This increased flexibility will reduce damage to the vascular walls while the sheath 20 is placed, and will allow larger medical devices to be placed within a catheter of given outside diameter. In some useful embodiments, the catheter wall has a thickness of less than about 0.010 inches as shown by the letter T in FIG. 4. In other useful embodiments, the catheter wall has a thickness of less than about 0.003 inches. In some particularly useful embodiments, the catheter wall has a thickness of less than about 0.0005 inches.

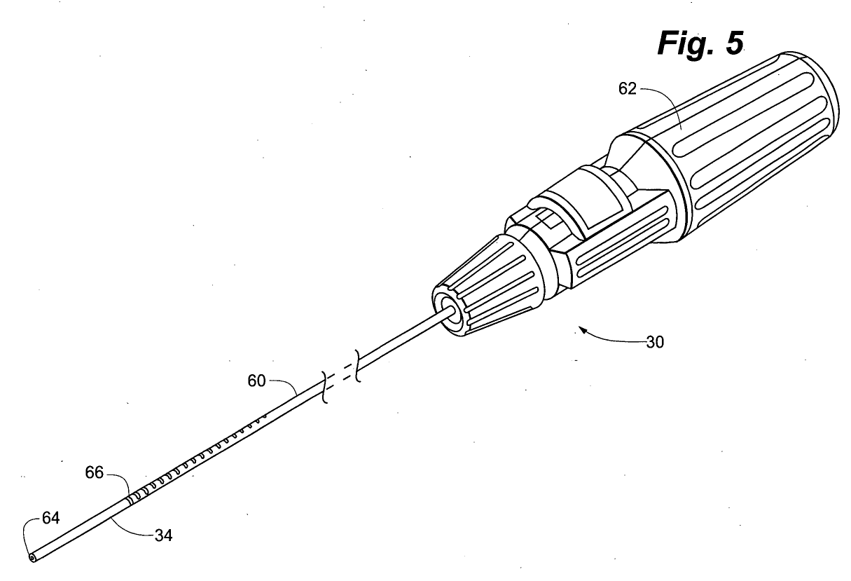

As mentioned above, the deployment actuator 40 may be used with an articulator 30 which has steerable technology, as shown in FIG. 5. The articulator 30 has a wire portion 60, a handle portion 62, and a core wire 64. By the manipulation of handle 62, the core wire 64 is extended or retracted within the wire portion 60. Such manipulation articulates the distal end 34 of the wire portion 60 to facilitate steering the articulator 30 through the body. In some embodiments, notches 66 are provided to promote articulation of the distal end 34. Such an articulator 30 is particularly suited to be used with a deployment actuator 40 because, in some embodiments, it allows at least part of the steerable distal end 34 to extend beyond the distal end of the sheath 20. Therefore, sheath 20 does not substantially interfere with the articulation of the distal end 34 and the steerability of the articulator 30 is not compromised.

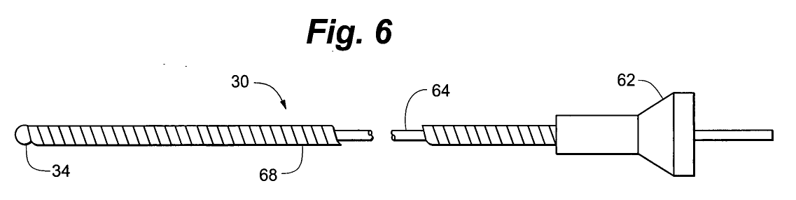

Also as mentioned above, the deployment actuator 40 may be combined with an adjustable stiffness articulator 30, as shown in FIG. 6. An adjustable stiffness articulator 30 comprises a stylet core wire 64, a space wound flat wire spring or compression member 68, and a handle 62. In some embodiments, as the handle 62 is pushed toward the distal end 34 thereby compressing compression member 68 and the overall flexibility of the articulator 30 begins to stiffen. Some embodiments of the invention provide for at least part of the distal end 34 to extend beyond the distal end of the sheath 20. Therefore, sheath 20 does not interfere with the distal end of the articulator 30 and does not compromise the benefits the adjustable stiffness feature provides in routing the device 10 through the body.

The present invention also includes a method of placing a medical device inside a body. In some embodiments, the method includes the steps of providing a device 10 having a medical device 12, such as a sheath 20, having a proximate end and a distal end, and an articulator 30 having a proximate end 32 and a distal end 34. The articulator 30 and the medical device 12 may be operatively connected proximate their distal ends. Further, a deployment actuator 40, such as a tube 42, may be provided. In some embodiments, an incision is made and the device 10 is inserted through the incision into a lumen of a mammalian body and routed through the body until in reaches a desired location. When it is desired to retract the articulator 30 while leaving the sheath 20 in place, they may be separated by applying an axial force to the deployment actuator 40 in either the distal and/or proximal directions. Thereafter, deployment tube 40 and articulator 30 may be retracted from the body.

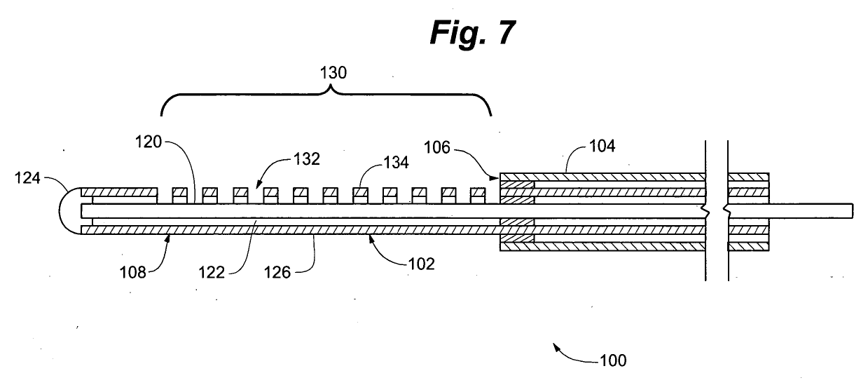

FIG. 7 is a cross sectional view of a device 100 comprising an articulator 102 and a sheath 104. A distal portion of sheath 104 is detachably attached to a distal portion of articulator 102 at a joint 106. In the embodiment of FIG. 7, articulator 102 comprises a first wire 108 and a second wire 120. With reference to FIG. 7, it will be appreciated that a portion of second wire 120 is disposed within a first lumen 122 defined by first wire 108. A distal portion of second wire 120 is fixed to a distal portion of first wire 108 at a tip member 124. Tip member 124 may comprise, for example, generally ball shaped mass of metal (e.g., solder) or other material.

First wire 108 comprises a wall 126 defining a lumen 122. In the embodiment of FIG. 7, a steerable portion 130 of first wire 108 comprises a portion of wall 126 that defines a plurality of slots 132. A rib 134 of steerable portion 130 of first wire 108 is defined by each adjacent pair of slots 132. Slots 132 may be positioned and dimensioned such that steerable portion 130 of first wire 108 can be urged to selectively assume various generally curved shapes. In some useful embodiments of the present invention, relative movement of the proximal end of second wire 120 relative to the proximal end of first wire 108 causes a steerable portion 130 of first wire 108 to assume a generally bent shape.

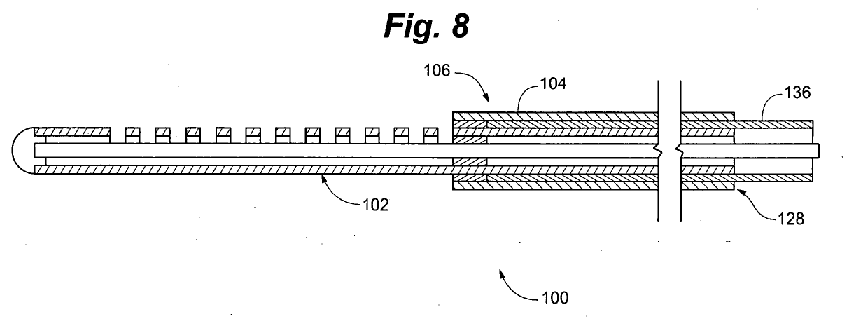

FIG. 8 is an additional cross sectional view of the device 100 shown in the previous figure. In the embodiment of FIG. 8, a tube 136 is disposed within lumen 128 defined by sheath 104. With reference to FIG. 8, it will be appreciated that tube 136 is disposed about articulator 102. A distal end of tube 136 is disposed proximate joint 106. In some useful methods in accordance with the present invention, tube 136 may be used to sever joint 106.

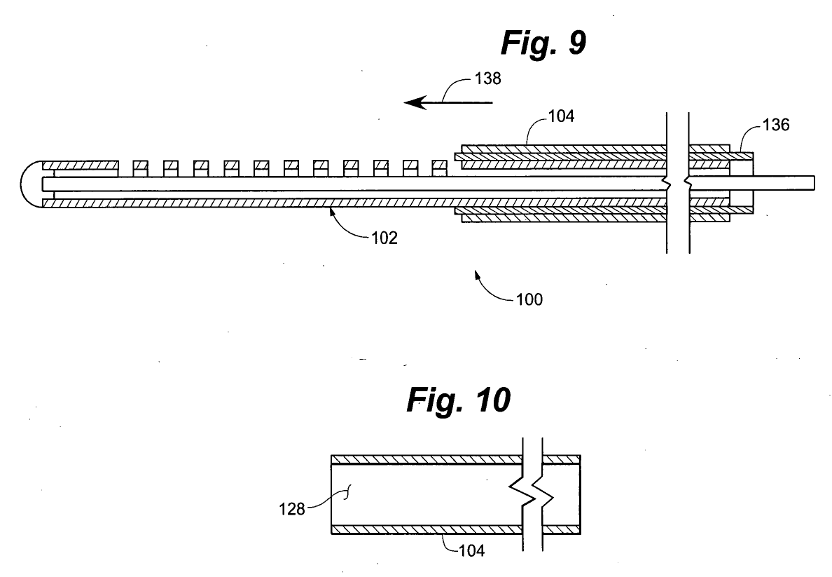

FIG. 9 is an additional cross sectional view of the device 100 shown in the previous figure. In the embodiment of FIG. 9, tube 136 has been urged in a distal direction 138 relative to articulator 102 and sheath 104. With reference to FIG. 9, it will be appreciated that tube 136 has severed the joint that had previously connected sheath 104 and articulator 102. In the embodiment of FIG. 9, articulator 102 and tube 136 are slidingly disposed in lumen 128 defined by sheath 104.

FIG. 10 is an additional cross sectional view of sheath 104 shown in the previous figure. In the embodiment of FIG. 10, tube 136 and articulator 102 have been withdrawn from lumen 128 defined by sheath 104. In some methods in accordance with the present invention, articulator 102 aids a surgeon in advancing sheath 104 into the body of a patient until the distal end of sheath 104 is disposed proximate a target region of the body. Also in some methods in accordance with the present invention, articulator 102 is withdrawn from lumen 128 defined by sheath 104 once the distal end of sheath 104 is disposed proximate a target region of the body. When this is the case, diagnostic and/or therapeutic devices may be advanced through lumen 128 defined by sheath 104.

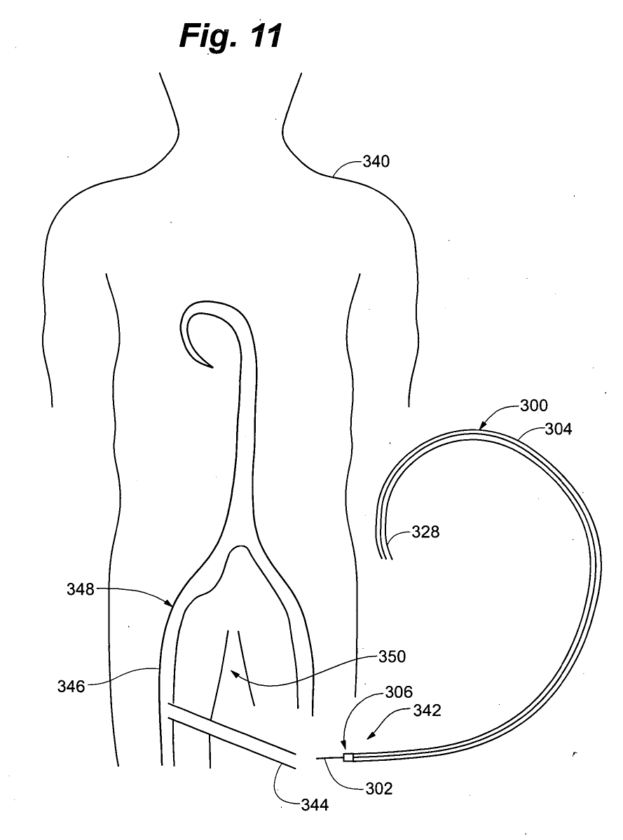

FIG. 11 is ventral view of a patient 340 and a device 300 in accordance with an exemplary embodiment of the present invention. Device 300 comprises a sheath 304 and the articulator 302. A distal portion 342 of sheath 304 is fixed to articulator 302 at a joint 306. In FIG. 11, an introducer 344 is positioned such that its distal end is positioned within a large blood vessel 346 of a vasculature 348 of patient 340.

Device 300 may be used to access remote regions of vasculature 348 of patient 340 to facilitate various medical procedures. For example, device 300 may be used to deliver diagnostic or therapeutic agents to target sites within the vasculature 348 of patient 340. By way of another example, device 300 may be used to deliver diagnostic or therapeutic devices to target sites within the vasculature 348 of patient 340.

It is contemplated that device 300 may be used in conjunction with an intravascular catheter to perform percutaneous transluminal coronary angioplasty (PCTA). When this is the case, the device may be advanced through the patient's vasculature until its distal tip is located proximate a restriction in a diseased vessel. In many cases, the device's path through the vascular system will be tortuous, requiring the device to change direction many times. Once the device is positioned, articulator 302 may be withdrawn from lumen 328 and a balloon catheter may be advanced into lumen 328 of device 300. The balloon catheter may be urged distally until a balloon fixed near the distal end of the catheter is centered on the restriction in the diseased vessel. The balloon may then be inflated to open the restriction. It is important to note that PTCA is just one example of the many procedures that may be facilitated by device 300.

A device in accordance with the present invention may also be use to facilitate endoscopic retrograde cholangio-pancreaticography (ERCP). ERCP procedures are often used when diagnosing and treating abnormal pathologies within the bile duct and the pancreatic duct. During such a procedure, device 300 may be inserted into a patients mouth guided through the patient's alimentary tract or canal until the distal end of the device is proximate the papilla of vater leading to the bile duct and the pancreatic duct. The device may then be guided through the orifice to the papilla of vater (located between the sphincter of oddi) leading to the bile duct and the pancreatic duct. The device may be advanced until the distal end of the catheter is positioned in a desired location. The wire may then be withdrawn from the lumen of the sheath. The sheath may be used to deliver fluoroscopic fluid to the bile duct and the pancreatic duct in order to diagnose pathological changes. A catheter may be advanced through the sheath. The catheter may also be used to take biopsies, extract stones or insert stents to provide for an unobstructed bile or pancreatic flow. Once the catheter is properly positioned, the sheath may help to maintain the position of the catheter during these procedures.

In FIG. 11, introducer 344 is shown extending into a large blood vessel 346 that is located near the groin 350 of patient 340. Large blood vessel 346 may be, for example, a vein or an artery. Device 300 may be advanced through vasculature 348 toward a target site in the body of the patient. Device 300 may be advanced until the distal end of sheath 304 is proximate the target site. Once the distal end of sheath 304 is proximate the target site, the connection between articulator 302 and sheath 304 may be severed. When this is the case, articulator 302 will be slidingly disposed within lumen 328 of sheath 304. Articulator 302 may then be withdrawn from lumen 328 of sheath 304.

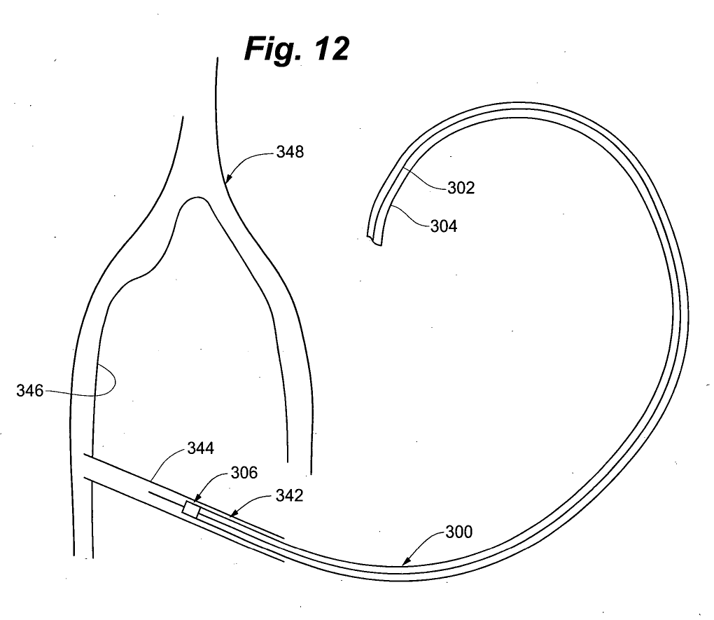

FIG. 12 is a diagrammatic view illustrating vasculature system 348 shown in the previous figure. In FIG. 12, an introducer 344 is positioned such that its distal end is positioned within a large blood vessel 346 of a vasculature 348 of patient 340. In the embodiment of FIG. 12, a distal portion of device 300 has been inserted into introducer 344. Device 300 comprises a sheath 304 and the articulator 302. A distal portion 342 of sheath 304 is fixed to articulator 302 at a joint 306.

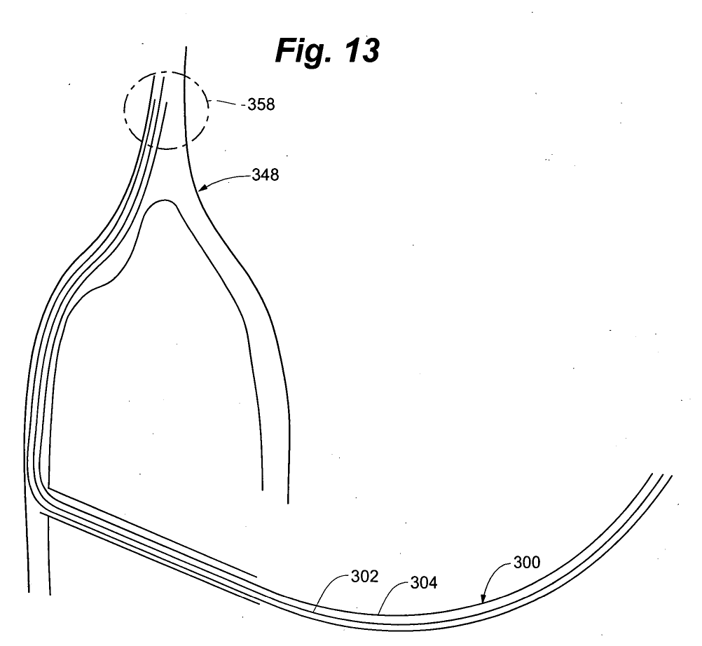

FIG. 13 is an additional diagrammatic view of device 300 and vasculature system 348 shown in the previous figure. In the embodiment of FIG. 13, device 300 has been advanced into vasculature 348 such that distal end 356 of sheath 304 is disposed proximate a target area 358 of vasculature 348. In the embodiment of FIG. 13, the joint that connected sheath 304 to articulator 302 has been severed.

In some methods in accordance with the present invention, articulator 302 and sheath 304 of device 300 are advanced into the vasculature 348 together. Once the distal end of sheath 304 is proximate a target site the connection between articulator 302 and sheath 304 may be severed. When this is the case, articulator 302 will be slidingly disposed within lumen 328 of sheath 304. Articulator 302 may then be withdrawn from lumen 328 of sheath 304.

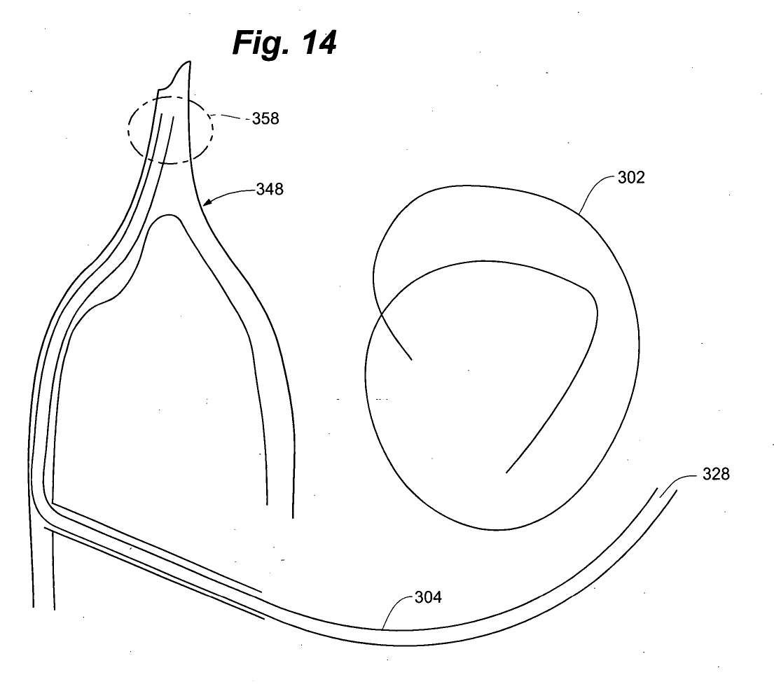

FIG. 14 is an additional diagrammatic view of sheath 304 and vasculature system 348 shown in the previous figure. In the embodiment of FIG. 14, the joint connecting sheath 304 and articulator 302 has been severed. Also in the embodiment of FIG. 14, articulator 302 has been withdrawn from lumen 328 of sheath 304. Sheath 304 may be used to access target area 358 of vasculature 348. For example, diagnostic and/or therapeutic agents may be advanced through lumen 328 to target area 358. By way of another example, diagnostic and/or therapeutic devices may be advanced through lumen 328 to target area 358.

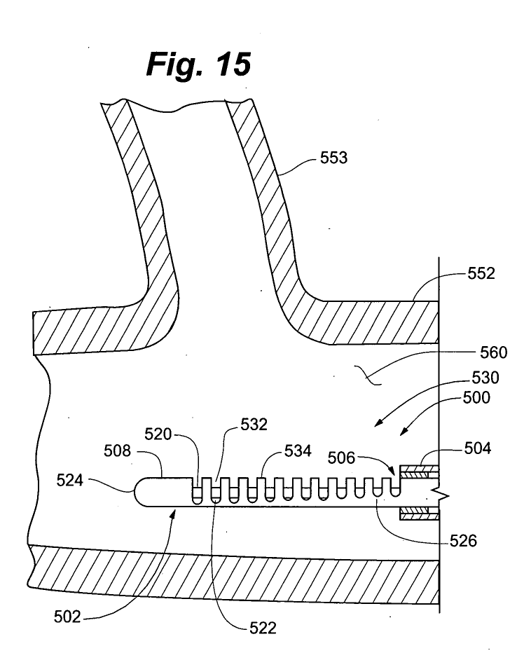

FIG. 15 is a partial cross sectional view of a device 500 comprising an articulator 502 and a sheath 504. In the embodiment of FIG. 15, device 500 is disposed within the interior 560 of a first blood vessel 552. The interior 560 of first blood vessel 552 communicates with the interior of a second blood vessel 553. A distal portion of sheath 504 is detachably attached to a distal portion of articulator 502 at a joint 506. In the embodiment of FIG. 15, articulator 502 comprises a first wire 508 and a second wire 520. With reference to FIG. 15, it will be appreciated that a portion of second wire 520 is disposed within a first lumen 522 defined by first wire 508. A distal portion of second wire 520 is fixed to a distal portion of first wire 508 at a tip member 524. Tip member 524 may comprise, for example, generally ball shaped mass of metal.

First wire 508 comprises a wall 526 defining a lumen 522. In the embodiment of FIG. 15, steerable portion 530 of first wire 508 comprises a portion of wall 526 that defines a plurality of slots 532. A rib 534 of steerable portion 530 of first wire 508 is defined by each adjacent pair of slots 532. Slots 532 may be positioned and dimensioned such that steerable portion 530 of first wire 508 can be urged to selectively assume various generally curved shapes. In some useful embodiments of the present invention, relative movement of the proximal end of second wire 520 relative to the proximal end of first wire 508 causes a steerable portion 530 of first wire 508 to assume a generally bent shape.

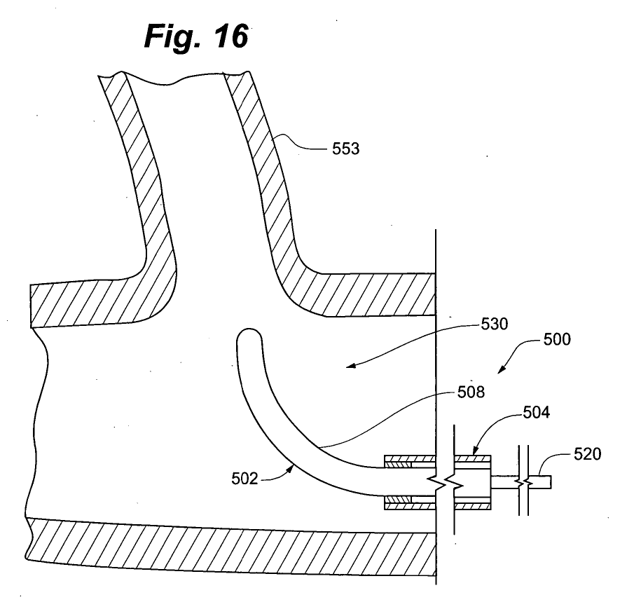

FIG. 16 is an additional cross sectional view of the device 500 shown in the previous figure. In the embodiment of FIG. 16, the proximal end of second wire 520 has been urged proximally relative to the proximal end of first wire 508. With reference to FIG. 16, it will be appreciated that a steerable portion 530 of first wire 508 is assuming a generally bent shape. In the embodiment of FIG. 16, urging steerable portion 530 of first wire 508 to assume the bent shape shown may assist a surgeon in advancing articulator 502 and sheath 504 into the interior of second blood vessel 553.

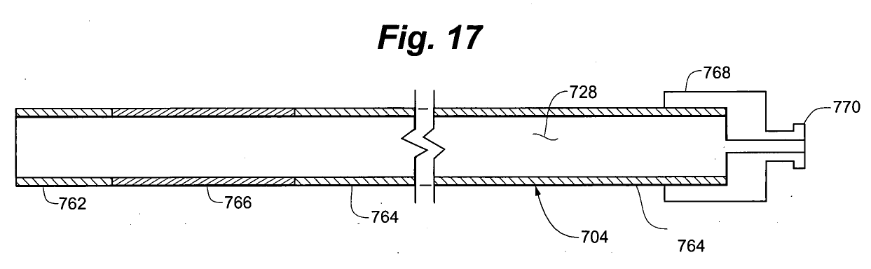

FIG. 17 is a cross sectional view of a sheath 704 in accordance with the present invention. In the embodiment of FIG. 17, sheath 704 comprises a distal segment 762, a proximal segment 764 and an intermediate segment 766 that is disposed between distal segment 762 and proximal segment 764. In the embodiment of FIG. 17, distal segment 762 has a greater durometer than proximal segment 764. Distal segment 762 may function to hold the distal end of sheath 704 proximate a target location in a human body. Distal segment 762 may also function to increase the strength of a connection between sheath 704 and an articulator. Distal segment 762 may be an elongated section of a few millimeters comprising a harder material.

In the embodiment of FIG. 17, a hub 768 is fixed to sheath 704 proximate it's proximal end. Hub 768 includes a luer fitting 770 that may be used to connect sheath 704 to other devices. Sheath 704 differs from existing catheters by virtue of a thin wall and flexibility. The thin wall may allow blood to flow more easily around and along the length of the sheath.

Sheath 704 may be formed, for example, using an extrusion process. Intermediate segment 766 of sheath 704 may comprise a mixture of the materials of distal segment 762 and proximal segment 764 to promote a strong bond between distal segment 762 and proximal segment 764. Proximal segment 764 of sheath 704 may have a hoop strength that is selected such that sheath 704 will collapse while it is disposed within a blood vessel. The blood pressure causes the sheath to collapse. The blood pressure may be for example 60 millimeters of mercury. The relatively thin, flexible wall of sheath 704 allows blood to flow more easily around and along the length of sheath 704. The wall of sheath 704 is soft enough that the arterial pressure will collapse the thin wall causing little occlusion to the blood flow. The sheath may remain collapsed until a secondary device is passed through the sheath lumen 728. The lumen will open to allow the device to pass but will still allow maximum blood flow.

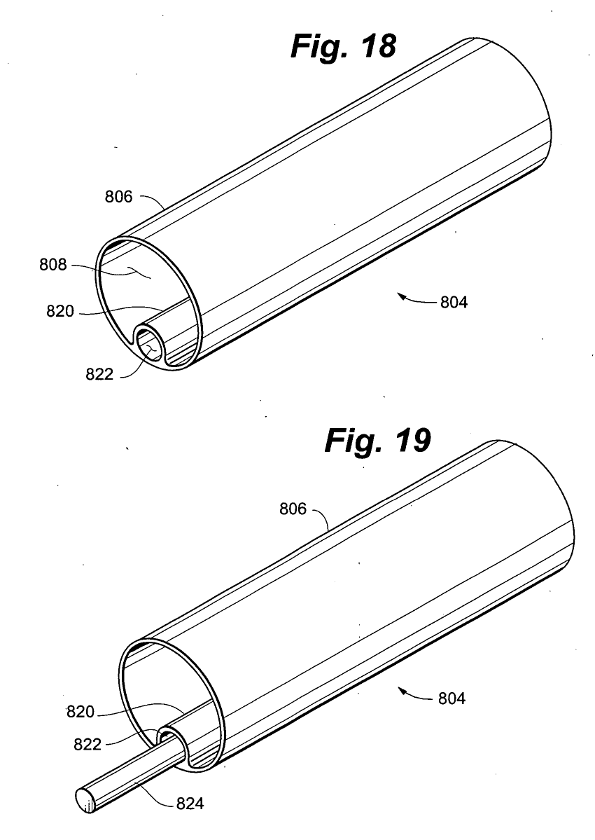

FIG. 18 is an isometric view of a sheath 804 in accordance with an exemplary embodiment of the present invention. Sheath 804 comprises a first wall 806 defining a first lumen 808. In FIG. 18, a second wall 820 of sheath 804 is shown substantially within first lumen 808 defined by first wall 806. Second wall 820 defines a second lumen 822. In some embodiments of the present invention, second lumen 822 is dimensioned to receive an articulator and first lumen 808 is large enough to allow a secondary device to pass therethrough.

FIG. 19 is an isometric view of an assembly including sheath 804 shown in the previous figure. In the embodiment of FIG. 19, an articulator 824 is shown extending through second lumen 822 defined by second wall 820. In some methods in accordance with the present invention, articulator 824 may be used to urge sheath 804 through a vessel (e.g., a blood vessel). In some useful embodiments of the present invention, first wall 806 of sheath 804 is capable of assuming various shapes. For example, first wall 806 may be urged to assume a somewhat collapsed shape while sheath 804 is urged through a vessel.

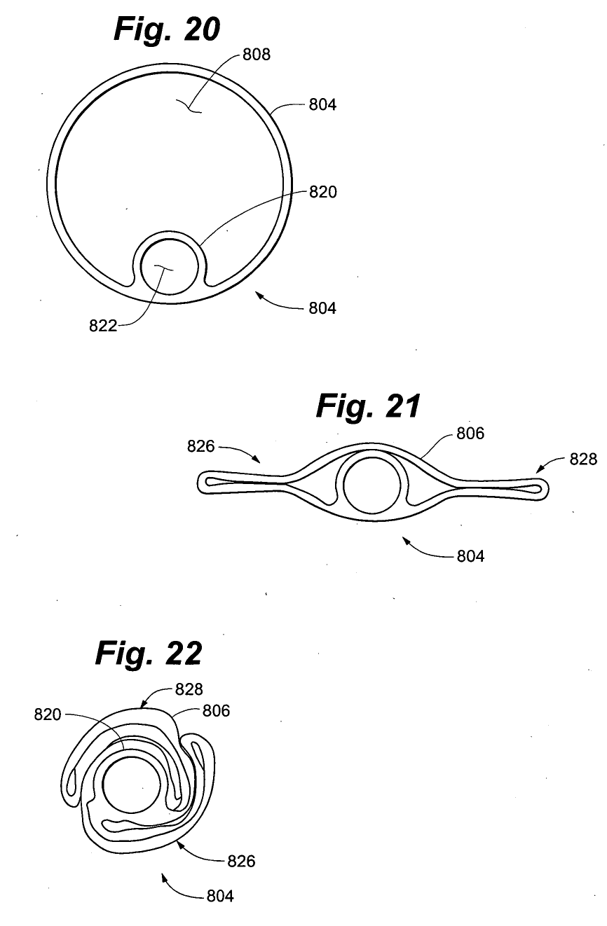

FIG. 20 is an axial view of sheath 804 shown in the previous figures. In FIG. 20, first wall 806 is shown assuming an expanded shape in which first wall 806 defines a first lumen 808 having a substantially cylindrical shape. In some embodiments of the present invention, first lumen 808 is large enough to allow a secondary device to pass through first lumen 808 while first wall 806 is assuming an expanded shape. Second lumen 822 defined by second wall 820 of sheath 804 is also visible in FIG. 20. In some embodiments of the present invention, second lumen 822 is dimensioned to receive an articulator that can be used to advance sheath 804 within a vessel.

FIG. 21 is an additional axial view showing sheath 804 shown in the previous figure. In the embodiment of FIG. 21, first wall 806 is assuming a somewhat collapsed shape. In the embodiment of FIG. 21, first wall 806 forms a first flap 826 and a second flap 828.

FIG. 22 is an additional axial view showing sheath 804 shown in the previous figure. In the embodiment of FIG. 22, first flap 826 and second flap 828 formed by first wall 806 are both wrapped around second wall 820. In some methods in accordance with the present invention, first wall 806 of sheath 804 may remain in a somewhat collapsed shape while sheath 804 is advanced into a blood vessel. Also in some methods in accordance with the present invention, first wall 806 may be urged to assume a more expanded shape as a secondary device is advanced though first lumen 808.

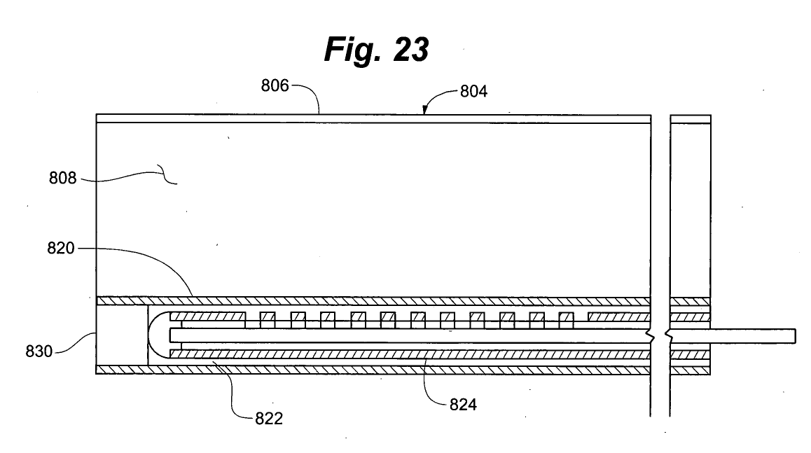

FIG. 23 is a cross-sectional view of a sheath 804 in accordance with an exemplary embodiment of the present invention. Sheath 804 comprises a first wall 806 defining a first lumen 808 and a second wall 820 defining a second lumen 822. In the embodiment of FIG. 23, an articulator 824 is disposed within second lumen 822. Also in the embodiment of FIG. 23, an end wall 830 extends across a distal end of second lumen 822. In some methods in accordance with the present invention, articulator 824 may push against end wall 830 to advance sheath 804 into a vessel.

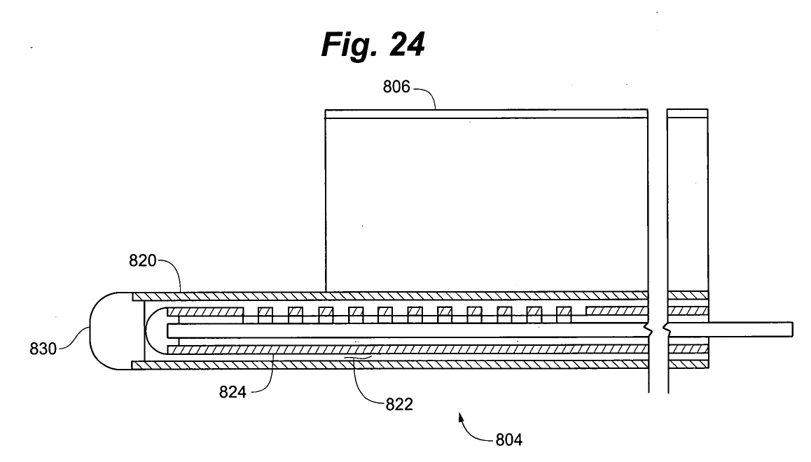

FIG. 24 is a cross-sectional view of a sheath 804 in accordance with an additional exemplary embodiment of the present invention. Sheath 804 comprises a first wall 806 and a second wall 820. With reference to FIG. 24, it will be appreciated that second wall 820 extends beyond a distal end of first wall 806. In the embodiment of FIG. 24, an articulator 824 is disposed in a second lumen 822 defined by second wall 820. Also in the embodiment of FIG. 24, an end wall 830 extends across a distal end of second lumen 822. In some methods in accordance with the present invention, articulator 824 may push against end wall 830 to advance sheath 804 into a vessel.

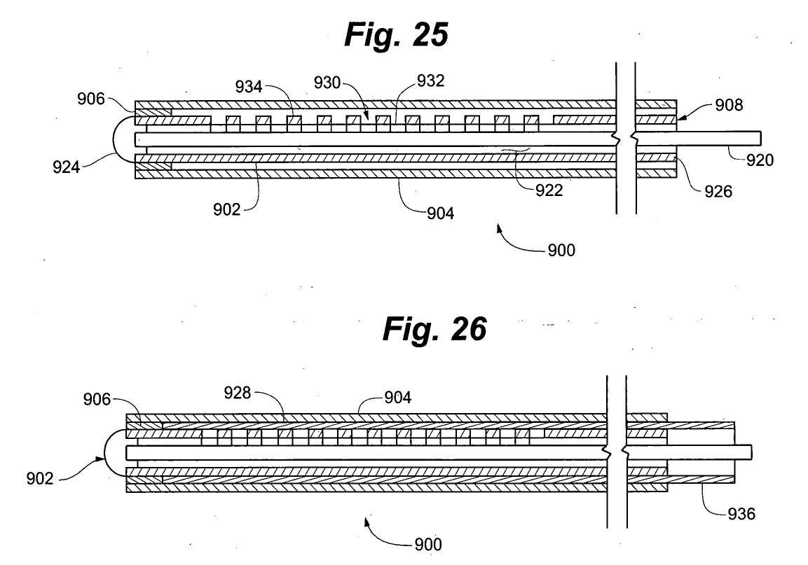

FIG. 25 is a cross sectional view of a device 900 comprising an articulator 902 and a sheath 904. A distal portion of sheath 904 is detachably attached to a distal portion of articulator 902 at a joint 906. In the embodiment of FIG. 25, articulator 902 comprises a first wire 908 and a second wire 920. With reference to FIG. 25, it will be appreciated that a portion of second wire 920 is disposed within a first lumen 922 defined by first wire 908. A distal portion of second wire 920 is fixed to a distal portion of first wire 908 at a tip member 924. Tip member 924 may comprise, for example, generally ball shaped mass of metal (e.g., solder) or other material.

First wire 908 comprises a wall 926 defining a lumen 922. In the embodiment of FIG. 25, a steerable portion 930 of first wire 908 comprises a portion of wall 926 that defines a plurality of slots 932. A rib 934 of steerable portion 930 of first wire 908 is defined by each adjacent pair of slots 932. Slots 932 may be positioned and dimensioned such that steerable portion 930 of first wire 908 can be urged to selectively assume various generally curved shapes. In some useful embodiments of the present invention, relative movement of the proximal end of second wire 920 relative to the proximal end of first wire 908 causes a steerable portion 930 of first wire 908 to assume a generally bent shape.

FIG. 26 is an additional cross sectional view of the device 900 shown in the previous figure. In the embodiment of FIG. 26, a tube 936 is disposed within lumen 928 defined by sheath 904. With reference to FIG. 26, it will be appreciated that tube 936 is disposed about articulator 902. A distal end of tube 936 is disposed proximate joint 906. In some useful methods in accordance with the present invention, tube 936 may be used to sever joint 906.

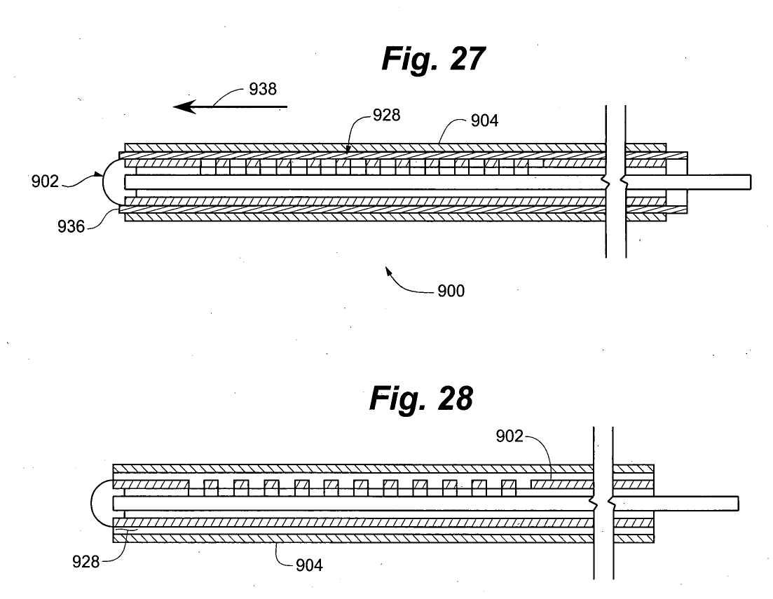

FIG. 27 is an additional cross sectional view of the device 900 shown in the previous figure. In the embodiment of FIG. 27, tube 936 has been urged in a distal direction 938 relative to articulator 902 and sheath 904. With reference to FIG. 27, it will be appreciated that tube 936 has severed the joint that had previously connected sheath 904 and articulator 902. In the embodiment of FIG. 27, articulator 902 and tube 936 are slidingly disposed in lumen 928 defined by sheath 904.

FIG. 28 is an additional cross sectional view of sheath 904 shown in the previous figure. In the embodiment of FIG. 28, tube 936 has been withdrawn from lumen 928 defined by sheath 904. With reference to FIG. 28, it will be appreciated that the joint that had previously connected sheath 904 and articulator 902 has been severed and articulator 902 is now slidingly disposed in lumen 928 defined by sheath 904.

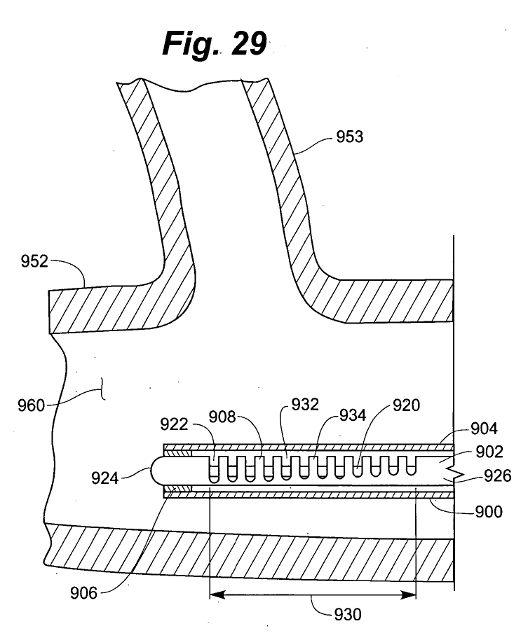

FIG. 29 is a partial cross sectional view of a device 900 comprising an articulator 902 and a sheath 904. In the embodiment of FIG. 29, device 900 is disposed within the interior 960 of a first blood vessel 952. The interior 960 of first blood vessel 952 communicates with the interior of a second blood vessel 953. A distal portion of sheath 904 is detachably attached to a distal portion of articulator 902 at a joint 906. In the embodiment of FIG. 29, articulator 902 comprises a first wire 908 and a second wire 920. With reference to FIG. 29, it will be appreciated that a portion of second wire 920 is disposed within a first lumen 922 defined by first wire 908. A distal portion of second wire 920 is fixed to a distal portion of first wire 908 at a tip member 924. Tip member 924 may comprise, for example, generally ball shaped mass of metal.

First wire 908 comprises a wall 926 defining a lumen 922. In the embodiment of FIG. 29, steerable portion 930 of first wire 908 comprises a portion of wall 926 that defines a plurality of slots 932. A rib 934 of steerable portion 930 of first wire 908 is defined by each adjacent pair of slots 932. Slots 932 may be positioned and dimensioned such that steerable portion 930 of first wire 908 can be urged to selectively assume various generally curved shapes. In some useful embodiments of the present invention, relative movement of the proximal end of second wire 920 relative to the proximal end of first wire 908 causes a steerable portion 930 of first wire 908 to assume a generally bent shape.

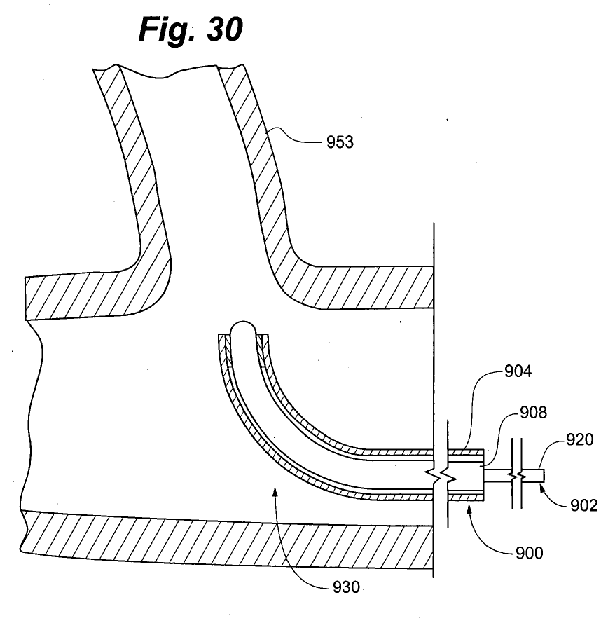

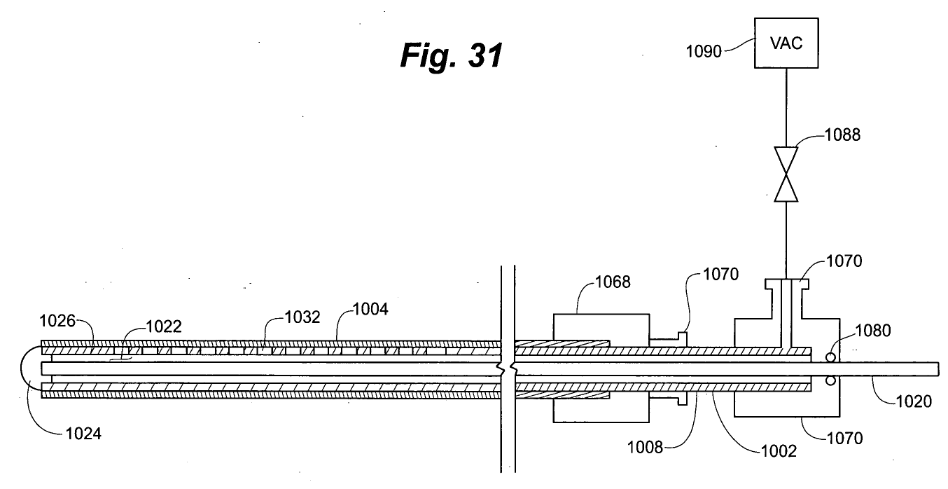

FIG. 30 is an additional cross sectional view of the device 900 shown in the previous figure. In the embodiment of FIG. 30, the proximal end of second wire 920 has been urged proximally relative to the proximal end of first wire 908. With reference to FIG. 30, it will be appreciated that a steerable portion 930 of first wire 908 is assuming a generally bent shape. With continuing reference to FIG. 30, it will be appreciated that a portion of sheath 904 that is disposed over steerable portion 930 of first wire 908 is also assuming a generally bent shape. In the embodiment of FIG. 30, sheath 904 to assume the bent shape shown may assist a surgeon in advancing articulator 902 and sheath 904 into the interior of second blood vessel 953.

FIG. 31 is a cross sectional view of a device in accordance with an additional embodiment of the present invention. The device of FIG. 31 includes a sheath 1004. A first hub 1068 is fixed to sheath 1004 proximate it's proximal end. First hub 1068 includes a luer fitting 1070 that may be used to connect sheath 1004 to other devices. In FIG. 31, an articulator is shown extending through first hub 1068 and a lumen defined by sheath 1004.

In the embodiment of FIG. 31, articulator 1002 comprises a first wire 1008 and a second wire 1020. First wire 1008 comprises a wall 1026 defining a lumen 1022. With reference to FIG. 31, it will be appreciated that a portion of second wire 1020 is disposed within the lumen 1022 defined by first wire 1008. A distal portion of second wire 1020 is fixed to a distal portion of first wire 1008 at a tip member 1024. In the embodiment of FIG. 31, wall 1026 of first wire 1008 defines a plurality of slots 1032.

A second hub 1070 is fixed to first wire 1008 proximate it's proximal end. Second hub 1070 includes a luer fitting 1070 that may be used to connect lumen 1022 of first wire 1008 to other devices. In the embodiment of FIG. 31, a vacuum source 1090 fluidly communicates with lumen 1022 of first wire 1008 via a valve 1088 and second hub 1070. In FIG. 31, second wire 1020 is shown extending through second hub 1070. A seal 1080 is disposed about second wire 1020.

In some methods in accordance with the present invention, a source of relatively low pressure (e.g., a vacuum source) may be used to selectively couple sheath 1004 to articulator 1002. For example, vacuum source 1090 may be used to make the internal pressure within the lumen of first wire 1008 lower than the external pressure outside of sheath 1004. This causes the external pressure to urge sheath 1004 against first wire 1008 of articulator 1002.

Numerous characteristics and advantages of the invention covered by this document have been set forth in the foregoing description. It will be understood, however, that this disclosure is, in many respects, only illustrative. Changes may be made in details, particularly in matters of shape, size and ordering of steps without exceeding the scope of the invention.

What is claimed is:

1. An apparatus, comprising:
a sheath having a proximate end, a distal end and a lumen extending therebetween;
an articulator disposed in the lumen of the sheath;
a distal portion of sheath being detachably attached to a distal portion of the articulator at a joint; and
a separator capable of severing the joint.

2. The system of claim 1, wherein the joint comprises an adhesive.

3. The system of claim 2, wherein the adhesive comprises a cyanoacrylate adhesive.

4. The system of claim 2, wherein the adhesive comprises a silicone adhesive.

5. The system of claim 2, wherein the adhesive comprises polyethylene glycol.

6. The system of claim 1, wherein the joint comprises a solvent welded bond.

7. The system of claim 1, wherein the joint comprises a thermally welded bond.

8. The system of claim 1, wherein the joint comprises a chemical bond.

9. The system of claim 1, wherein the joint comprises a mechanical linkage.

10. The system of claim 1, wherein the articulator is steerable.

11. The system of claim 1, wherein the separator comprises a tube.

12. The system of claim 1, wherein the separator comprises a wire disposed within a lumen of the tube.

13. The system of claim 12, wherein the wire extends proximally beyond a proximal end of the tube.

14. The system of claim 12, wherein the wire extends distally beyond a distal end of the tube.

15. The system of claim 1, wherein the separator is actuated at a proximate end thereof.

16. The system of claim 1, wherein a portion of the distal end of the deployment actuator extends beyond a portion of the distal end of the sheath, and a portion of the distal end of the articulator extends beyond a portion of the distal end of deployment actuator.

17. The system of claim 1, wherein the sheath comprises a lubricous coating on an outside surface thereof.

18. The system of claim 1, wherein the sheath comprises an anti-thrombic coating on an outside surface thereof.

19. The system of claim 1, wherein the sheath comprises a lubricous coating on an inside surface thereof.

20. The system of claim 1, wherein a distal region of the articulator has adjustable lateral stiffness.

21. The system of claim 1, wherein a distal region of the articulator is capable of a first shape having a first lateral stiffness and a second shape having a second lateral stiffness.

22. The system of claim 1, wherein the sheath has a lateral stiffness that is less than a lateral stiffness of the articulator.

23. The system of claim 1, wherein the sheath has a hoop strength selected such that a pressure within the body lumen causes the sheath to collapse.

24. A method comprising the steps of:
providing a device comprising a sheath disposed about an articulator with a distal portion of the sheath being fixed to the articulator at a joint;
inserting the device into a lumen of a body;
severing the joint; and
withdrawing the articulator from the sheath.

25. The method of claim 24, wherein the step of severing the joint comprises the step of applying an axial force to a deployment actuator.

26. The method of claim 25, wherein the axial force is applied to the deployment actuator in a distal direction.

27. The method of claim 24, wherein the deployment actuator comprises a tube.

28. A device comprising:
an articulator having a proximate end and a distal end;
a sheath having a proximate end and a distal end;
a distal portion sheath being detachably attached to a distal portion of the articulator at a joint; and
a separator capable of detaching the sheath from articulator.

29. A sheath comprising:
a first wall defining a first lumen;
a second wall defining a second lumen;
the second wall being substantially disposed within the first lumen;
the first wall being folded to form at least one flap;
the at least one flap being wrapped around the second wall.

30. An apparatus comprising:
a sheath having a proximate end, a distal end and a lumen extending therebetween;
an articulator disposed in the lumen of the sheath;
an internal pressure within a lumen of the articulator being lower than an external pressure outside of the sheath; and
the external pressure urging the sheath against the articulator for operatively connecting the sheath and the articulator.
